(19) United States
(12) Patent Application Publication
(10) Pub. No.: US 2022/0057391 A1
(43) Pub. Date: Feb. 24, 2022

(54) DIAGNOSTIC AND THERAPEUTIC FOR THE IDENTIFICATION AND TREATMENT OF SARS-CoV-2

(71) Applicant: FluoroMe, Inc., Houston, TX (US)

(72) Inventors: Dimitrios VLACHAKIS, Houston, TX (US); Charles Neblett, Houston, TX (US); Maria Kasti, Houston, TX (US)

(21) Appl. No.: 17/409,925

(22) Filed: Aug. 24, 2021

(30) Foreign Application Priority Data
Aug. 24, 2020 (GR) 20200100511
Aug. 24, 2020 (GR) 20200100512

(51) Int. Cl. G01N 33/543

(52) U.S. Cl. CPC G01N 33/54388; G01N 2333/165; G01N 2469/10

(57) ABSTRACT

The present invention provides methods and diagnostic kits for the detection or prediction of SARS-CoV-2. More particularly, the present invention provides for the diagnosis of SARS-CoV-2 by detecting in a human biological sample inhibition of protein translation by the phosphorylation of the eukaryotic initiation factor-2α (eIF-2α), and the formation of stress granules (SGs) known to be affected by Sars-CoV-2

### CROSS REFERENCE TO RELATED APPLICATIONS

This application claims the benefit of Greek patent application number 20200100511, filed Aug. 24, 2020, and Greek patent application number 20200100512, filed Aug. 24, 2020, each of which is incorporated herein by reference in its entirety.

### FIELD OF THE INVENTION

The present invention provides methods and diagnostic kits for the detection or prediction of SARS-CoV-2. More particularly, the present invention provides for the diagnosis of SARS-CoV-2 by detecting in a biological sample inhibition of protein translation by the phosphorylation of the eukaryotic initiation factor-2α (eIF-2α), and the formation of stress granules (SGs) known to be affected by Sars-CoV-2. In certain embodiments, the invention encompasses exosome-based diagnostics of infection and/or inflammation that are based on a novel exosome collection device from biological fluids such as urine or saliva in the form of a syringe, and a diagnostic test device that simultaneously detects exosomal biomarkers based on a quantitative assay and disease (infection/inflammation) biomarkers.

### BACKGROUND OF THE INVENTION

The outbreak of SARS-CoV-2, a virulent coronavirus that causes the COVID-19 disease, which has spread around the world with devastating effects in the human population. This disease follows the emergence of SARS-CoV and MERS-CoV in 2002 and 2012, respectively.

The biology of coronaviruses has been investigated over the past forty years leading to our current knowledge regarding virus entry routes, viral receptors, cellular and organ tropism (Rev Weiss and Leibowitz 2011, Weiss 2020). It has been recently shown that the current new coronavirus SARS-CoV-2 uses ACE2 receptors to invade host cells similarly as SARS-CoV (Zhou et al, 2020).

Clinical and epidemiological data from the Chinese CDC and regarding 72,314 case records (confirmed, suspected, diagnosed, and asymptomatic cases) were shared in the Journal of the American Medical Association (JAMA) (Feb. 24, 2020) and suggest a three scale severity disease: Mild disease: non-pneumonia and mild pneumonia; this occurred in 81% of cases. Severe disease: dyspnea, respiratory frequency ≥30/min, blood oxygen saturation (SpO2) ≤93%, PaO2/FiO2 ratio or P/F [the ratio between the blood pressure of the oxygen (partial pressure of oxygen, PaO2) and the percentage of oxygen supplied (fraction of inspired oxygen, FiO2)] <300, and/or lung infiltrates >50% within 24 to 48 hours; this occurred in 14% of cases.

Moreover, it has been recently published that high mortality in COVID-19 patients may be the result of virally driven hyperinflammation. Fatal cases of COVID-19 showed pathological patterns of secondary haemophagocytic lymphohistiocytosis (sHLH), which is an under-recognised, hyperinflammatory syndrome characterized by a fulminant and fatal hypercytokinaemia with multiorgan failure. In adults, sHLH is most commonly triggered by viral infections and occurs in 3.7-4.3% of sepsis cases. Cardinal features of sHLH include unremitting fever, cytopenias, and hyperferritinaemia; pulmonary involvement (including ARDS) occurs in approximately 50% of patients.

Currently, there are unrecognized patterns of the disease such as lymphopenia, hyper-ferritinemia leading perhaps to coagulation abnormalities and early hypoxemia with an initially preserved lung function. In any case the patterns of disease manifestation are variable and under investigation.

It has been suggested “influenza virus-cytokine-trypsin” cycle is one of the major underlying mechanisms of vascular hyperpermeability and multi-organ failure (MOF) in severe influenza. Severe influenza causes a marked increase in the levels of proinflammatory cytokines, such as tumor necrosis factor (TNF)-α, interleukin (IL)-6, and IL-1β. This potentially fatal immune response has been termed the cytokine storm. Such hypercytokinemia alters the cellular redox state through different cytokine receptors and reduces the expression of four complex I subunits, oxygen consumption and ATP synthesis in mitochondria, as well increasing mitochondrial O²⁻ production and the intracellular calcium concentration [Ca²⁺]

Importantly ATP depletion causes dissociation of zonula occludens-1, an intracellular tight junction component, from the actin cytoskeleton and thus increases junctional permeability. These cytokines also upregulate trypsin through activation of nuclear factor-kappa B (NF-κB) and activator protein 1 (AP-1), which mediates the post-translational proteolytic cleavage of viral envelope hemagglutinin (HA) and is crucial for viral entry and replication in various organs and endothelial cells. Trypsin also increases [Ca²⁺] and Cl⁻ and K⁺ secretion via the protease-activated receptor (PAR)-2, resulting in loss of zonula occludens-1 in endothelial cells and severe edema in the airways and colon. In addition, it has been reported that IAV infection induced degradation of glycogen synthase kinase (GSK)-3β-mediated β-catenin in adherens junctions of vascular endothelial cells, which resulted in increased membrane permeability.

These observations led to the conclusion that severe viral infections, e.g. induced by

Influenza A relate to an energy metabolic disorder.

Two sensitive mitochondrial enzymes, pyruvate dehydrogenase (PDH) in glucose oxidation and carnitine palmitoyltransferase (CPT) in long-chain fatty acid oxidation, have been reported to play key pathogenic roles in mitochondrial ATP crisis and MOF during severe IAV infection. It has been reported that severe IAV infection is associated with marked and specific upregulation of pyruvate dehydrogenase kinase (PDK), but not the related kinases PDKs1-3, in the skeletal muscle, liver, lungs, and heart, but not in the brain.

PDK4 phosphorylates PDH, a mitochondrial gatekeeper enzyme involved in glucose oxidation, and suppresses its activity, resulting in marked downregulation of glucose-mediated energy homeostasis, and culminating in ATP crisis. The expression of PDK4 is transcriptionally regulated by several nuclear hormone receptors that are activated in a tissue-specific manner by various metabolites and hormones, such as peroxisome proliferation-activated receptors (PPARs: PPARα, PPARβ/δ, and PPARγ), glucocorticoid receptors, estrogen-related receptors and thyroid hormone receptors.

These findings, together with the growing evidence for a link between cytokines and metabolic disorders, between cytokines and PPARs, and between PPARs and metabolic disorders through PDK4, have led to propose a new host cellular mechanism for MOF in severe influenza. This concept involves the “host metabolic disorders-cytokine” cycle, which links PPARs and PDK4, and in turn is closely related to the “influenza virus-cytokine-trypsin” cycle (H. Kido et al, 2016).

Exosomes are a class of extracellular vesicles (EVs) measuring 30-100 nm. These vesicles are produced and released by various types of cells, including epithelial cells, adipocytes and fibroblasts, cells of the nervous system including Schwann cells, astrocytes, and neurons, as well as in hematopoietic cells, where their secretion were detected for the first time. One of the main functions of exosomes is the maintenance of cellular homeostasis. These vesicles partake in the expulsion of harmful cellular constituents from cells, and deregulated exosome secretion may be a sign of a pathological condition (Takahashi et al., 2017). Moreover, exosomes are associated with the development and progression of several diseases, such as neurodegenerative diseases and cancer (Kang, 2020). Exosomal cargo, such as proteins and non-coding RNAs, are characteristic of the parent cell's physiological and pathophysiological condition. Specifically, diseased cells produce exosomes with different cargo than healthy cells. Thus, exosomal cargo could be a prime biomarker used to diagnose such pathological conditions (Lin et al., 2015a). Additionally, pathogens may hijack mechanisms of extracellular vesicle trafficking and assimilate viral components into exosomes (Crenshaw et al., 2018). Therefore, exosomes may also be used to detect possible viral and bacterial infections.

Exosome secretion has been reported for numerous cells in the nervous system, from neuronal cells to microglia and oligodendrocytes (Jan et al., 2017). These vesicles appear to have a great role in nervous system function since they participate in cell-to-cell communication and aid in synaptic plasticity regulation and the nerve regeneration process (Rastogi et al., 2021). Exosomes have been shown to contain infectious particles like prions. Prions promote the pathological refolding and aggravation of proteins which may lead to neurodegeneration. Thus, the identification of prion content in exosomes can help diagnose prion-related neurodegenerative diseases (Jan et al., 2017).

Exosomes carry many modulators of inflammation, such as microRNAs and cytokines. Moreover, exosomes can also cross the blood-brain barrier, allowing them to act as a communication channel between systemic inflammation and the central nervous system. A prime example of a neurodegenerative disease where inflammation plays a prominent role in neurodegeneration is multiple sclerosis. In experimental autoimmune encephalomyelitis, which is the inflammation-driven disease model of multiple sclerosis, pro-inflammatory cytokines promote exosome release, which themselves contain pro-inflammatory molecules, thus spreading inflammation (Soria et al., 2017). The identification and quantification of pro-inflammatory cytokines found in exosomes may help in multiple sclerosis diagnosis. Lastly, exosomes may contain proteins that are distinctive of a specific neurodegenerative disease, with a prime example being Alzheimer's Disease (AD). The main characteristics of Alzheimer's Disease are senile plaques of amyloid-beta (Aβ) peptide and neurofibrillary tangles caused by hyper-phosphorylated tau proteins. Recent studies suggest that the presence of tau in the cerebrospinal fluid (C SF) may act as a diagnostic biomarker for the early diagnosis of AD. AD patients display CSF and plasma exosomes which contain full-length tau, a feature absent in healthy people. Detecting exosomes with such cargo early on may help diagnose AD before its' clinical onset (Lakshmi et al., 2020).

The stability and presence of exosomes in most bodily fluids and their cargo's ability to describe the parent cell's physiological condition make these vesicles a promising liquid biopsy tool for cancer (Soung et al., 2017). It has been shown that cancer cells may secrete at least tenfold more exosomes than healthy cells (Li et al., 2017). Exosomes may harbor molecules characteristic of cancer cells which allow them to alter the tumor microenvironment and act on neighboring or distant cells, thus partaking in cancer development (Huang and Deng, 2019). Specifically, exosomes derived from cells of cancer patients harbor microRNAs that can initiate tumor growth in healthy cells. Consequently, using exosomes as biomarkers in early cancer diagnosis may involve detecting the presence of distinct mature miRNAs in bodily fluids without requiring an invasive tissue biopsy (Anastasiadou and Slack, 2014). Additionally, exosomal protein cargo can also be used for cancer diagnosis. Tumor-derived exosomes are enriched in immunosuppressive proteins in order to weaken anti-tumor immune responses (Whiteside, 2016). Therefore, the quantification of exosome cargo content of such proteins or proteins with known oncogenic attributes can be used for cancer diagnosis. Apart from their cargo, tumor-derived exosomes may also display tumor-specific surface markers, a characteristic which adds to their potential use as cancer biomarkers. It is not surprising, then, that there have been numerous clinical trials on various types of cancer such as breast cancer, pancreatic cancer, and lung cancer regarding their use as diagnostic tools (Makler and Asghar, 2020).

Exosomes have been implicated in several infectious diseases. Several viruses, including the human immunodeficiency virus 1 (HIV-1), hepatitis viruses such as hepatitis B virus (HVB) and hepatitis C virus (HCV), plus members of the human herpesvirus family, exploit exosome cargo selection mechanisms to promote viral transmission. HCV, as an example, makes use of the exosome biogenesis machinery to produce enveloped virions that help the virus avoid immune surveillance (Rodrigues et al., 2018). By identifying viral components harbored in exosomes, it is possible to better diagnose patients who display a low viral load (Crenshaw et al., 2018; Rodrigues et al., 2018). Bacterial infections have also been associated with exosomes. Mycobacterium tuberculosis which is the causative agent of tuberculosis has been associated with exosome trafficking (Rodrigues et al., 2018). M. tuberculosis is inhaled into the lungs through the trachea and is later engulfed by alveolar macrophages. Once inside a macrophage, M. tuberculosis is captured into phagosomes, whose goal is to deliver their cargos to lysosomes for degradation. Nonetheless, in many cases, M. tuberculosis may block the acidification and maturation of phagosomes and promote its' survival in the host macrophage (Chai et al., 2018). Exosomes produced from such infected cells potentially contain mycobacterial components, which may act as cell attractants for other macrophages. Moreover, the composition of exosomes may vary based on infection time (Wang et al., 2019). Therefore, quantifying mycobacteria components found in exosomes may help not only diagnose diseases such as tuberculosis but estimate the infection time too.

The above showcase that exosomes may help diagnose a wide variety of diseases. Moreover, their ability to remain stable in bodily fluids makes them an excellent disease biomarker (Boukouris and Mathivanan, 2015). The biggest hurdles in the use of exosomes as a diagnosis method are the current methods used for their isolation and analysis. There is no gold standard for exosome isolation, and their characterization remains difficult due to their somewhat undefined nomenclature (Ludwig et al., 2019). New technologies, though, are expected to provide more efficient, quick, and cost-effective exosome isolation methods. The increased interest in exosome research will also help elucidate these vesicles' nomenclature, the mechanisms underlying their function, and lay the foundation for the standardization of exosome isolation methods. Hence, exosomes can be considered a highly promising diagnostic tool in the forthcoming future.

Exosomes have been detected in many different types of biological fluids, such as blood, breast milk, urine, semen, amniotic fluid, saliva, bronchoalveolar lavage, and cerebrospinal fluid (Isola and Chen, 2017). In addition to the normal function of exosomes, their involvement in a variety of pathological conditions and the development of various diseases, including neurodegenerative diseases, liver disease, heart failure and cancer, has been recognized. Numerous studies have shown that pathogens have the ability to exploit the release of exosomes to infect host cells, thus avoiding the response of the host immune system (Ludwig et al., 2019).

According to experimental studies, the cargos and number of exosomes produced are altered by environmental factors or pharmacological treatments. So, the concentration and molecular cargo of exosomes isolated from blood or other type of biological fluids of patients with several diseases are modified in pathological conditions. Increased levels of circulating exosomes have been recorded in the blood of patients with different types of cancer, where the detection of tumor-specific proteins in the cargo of circulating exosomes leads to the conclusion that these exosomes are derived from cancer cells (Skog et al., 2008).

More specifically, in the case of cancer the biogenesis of exosomes is enhanced. Studies have shown that cancer cells produce and secrete a higher quantity of exosomes than normal proliferating cells, and increased levels of exosomes in plasma and other body fluids of cancer's patients are observed. Stress and hypoxia prevailing in the tumor microenvironment (TME) have been suggested as possible causes of increased exosome secretion by cancer cells. At the same time, p53 and heparanase, an enzyme overexpressed in many cancer cell-lines, are two proteins that are shown to regulate the increased production and secretion of exosomes by cancer cells. According to knock down studies, the role of Rap GTPase proteins, especially Rab27a and Rab27b, in controlling the secretory pathways strongly involved in exosome release has been documented, as a reduction in these proteins leads to a decrease in exosomes secretion from cancer cells. However, the mechanisms regulating exosomes secretion by cancer cells are not yet known (Whiteside, 2016).

Logozzi et al. studied the number of exosomes derived from a tumor in a mouse model and identified a correlation of exosome levels with tumor size. Clinical trials in patients with non-small cell lung cancer (NSCLC), esophageal cancer and ovarian cancer showed elevated levels of exosomes in the patients' plasma, comprising an indicator of poor prognosis (Shen et al., 2020). In addition, an increasing number of studies have shown that exosomes secretion and cargo are affected by new and conventional cancer therapies. According to the study of Keklikoglou et al., cytotoxic chemotherapy in cases of breast cancer caused an increase in exosome production, while increasing the levels of annexin A6 in exosomes and promoting the formation of a pre-metastatic niche caused by exosomes (Keklikoglou et al., 2019).

Exosome levels also increase during viral infection. This change is likely due to altered cellular activity of infected cells and the use of intracellular pathways of host cells by pathogens. More specifically, an example is the case of patients who have been infected by Plasmodium and have had symptoms for more than 6 days, in which 20-30% more exosomes derived from their platelets were detected. In another study of rotavirus (RV) cell infection, elevated levels of heat shock cognate protein 70, TGF-β1, and other exosome proteins were observed, reflecting the increased release of exosomes from human intestinal epithelial cells. In addition, in cells infected with the Ebola virus, the presence of viral matrix protein viral protein 40 (VP40) leads to the upregulation of exosome markers, as CD63, apoptosis-linked-gene-2 product-interacting protein X (Alix) and Endosomal Sorting Complex Required for Transport machinery-II proteins, suggesting the activation of exosomes biogenesis during EBOV infection. Similar effects have been observed in other diseases, such as HIV infection. In this case, an increase in exosomes' levels in patients' plasma compared to healthy ones was recognized and plasma-derived exosomes of HIV patients contained proteins related to immune activation and oxidative stress (Chettimada et al., 2018). An increase in exosomes production and altered cargo has been also reported after treatment with antiretroviral drugs (DeMarino et al., 2018). Moreover, in cases of acute lung injury, acute renal failure, acute myocardial damage or sepsis, increased levels of circulating exosomes as well as an alteration in their molecular cargo have been observed (Terrasini and Lionetti, 2017).

In the case of neurodegenerative diseases, abnormal protein exchange is observed due to changes in the number and content of exosomes. Alzheimer's disease has been associated with the accumulation of microtubule-associated cytosolic protein tau and its subsequent secretion into the extracellular space, where it is enclosed in exosomes. Moreover, astrocytes exposed to an amyloid peptide secrete active exosomes whose cargo consists of prostate apoptosis response 4 (PAR-4) and ceramide. These exosomes are taken from neighboring astrocytes and cause apoptosis (Sampey et al., 2014). In general, all neurodegenerative diseases are characterized by a common molecular mechanism involving the accumulation of proteins and the formation of inclusion bodies in specific areas of the nervous system. Exosomes' involvement in the spread of “injurious” proteins in neurodegenerative disorders has been demonstrated, as the accumulated proteins are removed from the neurons by processing them by endosomal pathway leading to either degradation into lysosomes or release as exosomes. In the study by Vella et al., the transfer of the misfolded pathogenic prion protein (PrPsc) associated with the exosomes to normal cells containing normal prion protein (PrP) was observed, suggesting a mechanism observed in neurodegenerative diseases that proteins tend to seed own aggregation via exosomes (Kalani et al., 2014).

It has been reported recently that around 30 SARS-COV-2 proteins interact with proteins of human mitochondrial metabolism and over 25 with components of electron transport chain (Gordon D E et al.,2020). Components of mitochondrial metabolism (e.g. pyruvate dehydrogenase) and/or activity of electron transport chain (i.e. Complex I, Complex IV) activity may be used as biomarkers for predicting the severity and prognosis of Covid19. Comorbidities associated with impaired mitochondrial function and loss of Δψm (e.g. diabetes) render innate immunity against SARS-COV-2 inadequate to mount efficient antiviral response, hence rendering those patients particularly susceptible to severe forms of the disease.

Working with novel models of Neuroinflammation (EAE) we know that Hypoxia is a key event in Neuroinflammation both cytotoxic and true Hypoxia as oxygen supply can reverse neurological deficit. In this model of Neuroinflammation (EAE) the cascade of inflammatory events leads to the expression of HIF-1a as expected but also to arrest of protein translation counteracting likely the consequences of energy insufficiency resulting from hypoxia and inflammation. (Unpublished data M. Kasti, Experimental Neuroinflammation A study of hypoxia and protein translation—discovery Thesis UCL 2013).

It is well known that one of the most important protective mechanisms to counteract cellular stress including viral induced stress is the phosphorylation of the eukaryotic translation initiation factor eIF-2α, by the endoplasmic reticulum resident kinase PERK, which is a part of a process known as the unfolded protein response (UPR).

### SUMMARY OF THE INVENTION

The invention encompasses a diagnostic immunoassay for the detection and treatment of

SARS-CoV-2 in a subject, said assay comprising the steps of:

(a) collecting a biological sample from a subject that is suspected of being infected with SARS-CoV-2; and

(b) detecting the concentration of eukaryotic initiation factor-2α (eIF-2α) in said sample by an immunological means;

wherein a reduced concentration of eIF-2α in said sample, as compared to the eIF-2α concentration in a plasma sample from a healthy mammal is predictive of SARS-CoV-2.

In certain embodiments, the subject is a mammal, preferably a human.

In certain embodiments, the detecting comprises the binding of eIF-2α by an antibody that specifically binds eIF-2α.

In certain embodiments, the immunological means uses a monoclonal antibody.

In certain embodiments, the antibody is labeled and said detecting comprises detecting the amount of label associated with said eIF-2α/antibody complex.

In certain embodiments, the label is selected from the group consisting of a radioactive label, an enzymatic label, a colorimetric label, and a fluorescent label.

In certain embodiments, the detecting comprises:

contacting said antibody with a labeled second antibody that binds said antibody; and

detecting the amount of labeled second antibody associated with said antibody immobilized on a substrate.

In certain embodiments, the detecting comprises a competitive assay, said assay comprising the steps of:

(a) providing a first antibody that specifically binds eIF-2α;

(b) contacting said first antibody with a labeled eIF-2α and said sample; and

(c) detecting the amount of labeled eIF-2α that forms an eIF-2α/antibody complex.

In certain embodiments, the first antibody is immobilized on a solid substrate.

In certain embodiments, the detecting comprises detecting the labeled eIF-2α bound to said first antibody.

In certain embodiments, the detecting comprises detecting the labeled eIF-2α remaining unbound in solution.

In certain embodiments, the detecting comprises Western Blotting utilizing an antibody that specifically binds eIF-2α.

In certain embodiments, the phosphorylation of eIF-2α, induces an immediate, but reversible arrest of protein synthesis.

Without being bound by theory, it is considered to be a part of a eIF-1α-independent response to hypoxia.

In certain embodiments, the adaptive mechanisms for counteracting hypoxia and energy insufficiency is a drastic decline in ATP consumption, leading to severe down-regulation of energy turnover by switching off certain parts of cell metabolism, and also using the available ATP more efficiently than during normoxic conditions. In certain embodiments, a similar mechanism is present in severe infection from Sars-CoV-2.

In certain embodiments, the proposed model of an energy metabolic disorder mentioned above enhances the conclusions that severe SARS-CoV-2 infection can lead to a metabolic disorder previously unrecognized sooner or later in the disease process.

Another enzyme that is phosphorylated and inactivated during torpor is pyruvate dehydrogenase (PDH). This enzyme complex converts pyruvate to acetyl CoA, thereby controlling aerobic oxidation of carbohydrates in the mitochondrial tricarboxylate acid cycle. PDH activity is controlled by several allosteric effectors and by covalently bound kinases and phosphatases that, respectively, phosphorylate (inactivate) and dephosphorylate (reactivate) the PDH complex. Dramatic reductions in aerobic carbohydrate oxidation during torpor have been explained by PDH kinase mediated phosphorylation of the PDH complex.

In certain embodiments, similar observations of upregulation of PDH is proposed in the influenza A metabolic disorder, which makes us suspect a common underlying mechanism of saving energy.

In certain embodiments, the results suggest that the current fatal viral disease induces for several reasons low oxygen concentration which could compromise energy homeostasis because it would not only diminish mitochondrial electron transport and ATP synthesis, but also activate AMP-activated protein kinase (AMPK). This activation down-regulates processes that consume oxygen, including the Na+/K+-ATPase and global protein synthesis allowing a metabolic adaptation to decreased oxygen levels.

In certain embodiments, the downstream effect of severe hypoxia can lead to a metabolic disorder which has not been recognized so far and induces a number of symptoms first time seen worldwide.

The metabolic adaptation designed to limit energy expenditure includes the inhibition of protein translation by the phosphorylation of the eukaryotic initiation factor (eIF-2a), and the formation of stress granules (SGs) already known to be affected by Sars-CoV-2.

In another embodiment, the invention is directed to an innovative exosome-based diagnostic platform for infection and/or inflammation diagnosis.

In certain embodiments, the platform is based on a novel exosome collection device from biological fluids such as urine and saliva following the principles of sequential filtration. The collection procedure uses a syringe-like device of 200 ml volume that consists of a biological sample compartment with a maximum volume of 50 ml and three sequential filters connected with two springs (FIG. 1). A 0.8 micrometer filter with pores at the level of 150 ml prevents cell and cell fragment flow, a 0.22 micrometer filter at the level of 100 ml prevents large vesicles, microorganisms and protein aggregations flow, and a at the level of 50 ml allows for remaining debris, but not exosome, flow. Upon pressure with a syringe plunger, the 0.8 micrometer filter and the 0.22 micrometer filter move towards the immobilized 500 kiloDalton filter and the biological sample is filtered out of debris. Exosomes are concentrated in the compartment between the 0.22 micrometer filter and 500 kiloDalton filter and can be collected by an insulin needle through an available outlet on the side walls of the syringe.

In another embodiment, the invention describes a diagnostic test device that simultaneously detects exosomal biomarkers based on a quantitative assay and disease (infection/inflammation) biomarkers. The invention is directed to a rapid, non-invasive detection system for diagnostics with higher accuracy, through a diagnostic test device that detects disease-specific biomarkers and, at the same time, quantifies the levels of disease-related exosomes and determines the severity and/or progression of the disease. The invention encompasses a rapid diagnostic test device that consists of three main parts (FIG. 2). A visual read-out for exosome qualitative and quantitative identification based on a quantitative lateral flow assay (LFA), a visual read-out for disease (infection/inflammation) biomarkers detection based on lateral flow immunoassay (LFIA) tests, and a sample pad for the application of urine or saliva samples, or collected liquid with concentrated exosomes from the exosome collection device.

### BRIEF DESCRIPTION OF THE DRAWINGS

FIG. 1 illustrates an exemplary SDS gel (left) and western blot (right, anti-His-tag antibody, penta-His conjugated) for the HCV helicase protein.

FIG. 2 illustrates FIG. 2 a graphical representation of the data set forth in table 2.

FIG. 3 illustrates a novel exosome isolation kit in the form of a syringe that collects exosomes from biological fluids such as urine and saliva.

FIG. 4 illustrates an innovative portable rapid diagnostic test that simultaneously detects exosome biomarkers based on a quantitative assay and disease (infection/inflammation) biomarkers.

### DETAILED DESCRIPTION OF THE INVENTION

Details of one or more embodiments of the presently disclosed subject matter are set forth in the accompanying description below. Other features, objects, and advantages of the presently disclosed subject matter will be apparent from the detailed description, figures, and claims. All publications, patent applications, patents, and other references disclosed herein are incorporated by reference in their entirety. Some of the polypeptides disclosed herein are cross-referenced to public database accession numbers. The complete sequences cross-referenced in the database are expressly incorporated by reference as are equivalent and related sequences present in other public databases. Also expressly incorporated herein by reference are all annotations present in the database associated with the sequences disclosed herein. In case of conflict, the present specification, including definitions, will control. Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

The terms “a,” “an,” and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments, ±10%, in some embodiments ±5%, in some embodiments ±1%, in some embodiments ±0.5%, and in some embodiments ±0.1% from the specified amount, as such variations are appropriate to perform the disclosed method.

Biological fluids are valuable as indicators of a subject's well-being and can be analyzed for data indicative of the presence or absence and progression of disease. For example, urine is one biological fluid that has clinical diagnostic value (Snyder & Pendergraph, 2005). In addition to low molecular weight species like glucose, bilirubin, ketones, sodium, potassium, and nitrites, urine contains specific proteins and peptides that have significant diagnostic value. One problem with the development of diagnostic protein or peptide markers (biomarkers) is the relative (low) concentration of the species that is sensitive and specific for a given disease; especially for the detection of a disease in the pre-pathologic state.

Considerable effort has been applied toward pre-fractionation of biological fluid samples with the goal of increasing the relative concentration of all peptide species in a given sample fraction (Anderson & Hunter, 2005; Vidal et al., 2005). Certain tissues through normal biological processes produce membrane vesicles containing a variety of polypeptides. In certain disease states, particular systems, such as for example the immune system can increase production of membrane vesicles.

The terms “polypeptide,” “protein,” and “peptide,” which are used interchangeably herein, refer to a polymer of the 20 protein amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted. The terms “protein”, “polypeptide” and “peptide” are used interchangeably herein when referring to a gene product. Thus, exemplary polypeptides include gene products, naturally occurring proteins, homologs, orthologs, paralogs, fragments and other equivalents, variants, and analogs of the foregoing.

The terms “polypeptide fragment” or “fragment”, when used in reference to a reference polypeptide, refers to a polypeptide in which amino acid residues are deleted as compared to the reference polypeptide itself, but where the remaining amino acid sequence is usually identical to the corresponding positions in the reference polypeptide. Such deletions can occur at the amino-terminus or carboxy-terminus of the reference polypeptide, or alternatively both. Fragments typically are at least 5, 6, 8 or 10 amino acids long, at least 14 amino acids long, at least 20, 30, 40 or 50 amino acids long, at least 75 amino acids long, or at least 100, 150, 200, 300, 500 or more amino acids long.

A fragment can retain one or more of the biological activities or diagnostic characteristics of the reference polypeptide. In some embodiments, a fragment can comprise a domain or feature, and optionally additional amino acids on one or both sides of the domain or feature, which additional amino acids can number from 5, 10, 15, 20, 30, 40, 50, or up to 100 or more residues. Further, fragments can include a sub-fragment of a specific region, which sub-fragment retains a function of the region from which it is derived.

“Synthetic oligonucleotide” refers to molecules of nucleic acid polymers of 2 or more nucleotide bases that are not derived directly from genomic DNA or live organisms. The term synthetic oligonucleotide is intended to encompass DNA, RNA, and DNA/RNA hybrids that have been manufactured chemically, or synthesized enzymatically in vitro.

An “oligonucleotide” is a nucleotide polymer having two or more nucleotide subunits covalently joined together. Oligonucleotides are generally about 10 to about 100 nucleotides. The sugar groups of the nucleotide subunits may be ribose, deoxyribose, or modified derivatives thereof such as OMe. The nucleotide subunits may be joined by linkages such as phosphodiester linkages, modified linkages or by non-nucleotide moieties that do not prevent hybridization of the oligonucleotide to its complementary target nucleotide sequence. Modified linkages include those in which a standard phosphodiester linkage is replaced with a different linkage, such as a phosphorothioate linkage, a methylphosphonate linkage, or a neutral peptide linkage. Nitrogenous base analogs also may be components of oligonucleotides in accordance with the invention.

A “target nucleic acid” is a nucleic acid comprising a target nucleic acid sequence. A “target nucleic acid sequence,” “target nucleotide sequence” or “target sequence” is a specific deoxyribonucleotide or ribonucleotide sequence that can be hybridized to a complementary oligonucleotide.

An “oligonucleotide probe” is an oligonucleotide having a nucleotide sequence sufficiently complementary to its target nucleic acid sequence to be able to form a detectable hybrid probe: target duplex under high stringency hybridization conditions. An oligonucleotide probe is an isolated chemical species and may include additional nucleotides outside of the targeted region as long as such nucleotides do not prevent hybridization under high stringency hybridization conditions. Non-complementary sequences, such as promoter sequences, restriction endonuclease recognition sites, or sequences that confer a desired secondary or tertiary structure such as a catalytic active site can be used to facilitate detection using the invented probes. An oligonucleotide probe optionally may be labelled with a detectable moiety such as a radioisotope, a fluorescent moiety, a chemiluminescent, a nanoparticle moiety, an enzyme or a ligand, which can be used to detect or confirm probe hybridization to its target sequence. Oligonucleotide probes are preferred to be in the size range of from about 10 to about 100 nucleotides in length, although it is possible for probes to be as much as and above about 500 nucleotides in length, or below 10 nucleotides in length.

A “hybrid” or a “duplex” is a complex formed between two single-stranded nucleic acid sequences by Watson-Crick base pairings or non-canonical base pairings between the complementary bases. “Hybridization” is the process by which two complementary strands of nucleic acid combine to form a double-stranded structure (“hybrid” or “duplex”). A “fungus” or “yeast” is meant any organism of the kingdom Fungi, and preferably, is directed towards any organism of the phylum Ascomycota.

“Complementarity” is a property conferred by the base sequence of a single strand of DNA or RNA which may form a hybrid or double-stranded DNA:DNA, RNA:RNA or DNA:RNA through hydrogen bonding between Watson-Crick base pairs on the respective strands. Adenine (A) ordinarily complements thymine (T) or uracil (U), while guanine (G) ordinarily complements cytosine (C).

The term “stringency” is used to describe the temperature, ionic strength and solvent composition existing during hybridization and the subsequent processing steps. Those skilled in the art will recognize that “stringency” conditions may be altered by varying those parameters either individually or together. Under high stringency conditions only highly complementary nucleic acid hybrids will form; hybrids without a sufficient degree of complementarity will not form. Accordingly, the stringency of the assay conditions determines the amount of complementarity needed between two nucleic acid strands forming a hybrid. Stringency conditions are chosen to maximize the difference in stability between the hybrid formed with the target and the non-target nucleic acid.

With “high stringency” conditions, nucleic acid base pairing will occur only between nucleic acid fragments that have a high frequency of complementary base sequences (for example, hybridization under “high stringency” conditions, may occur between homologs with about 85-100% identity, preferably about 70-100% identity). With medium stringency conditions, nucleic acid base pairing will occur between nucleic acids with an intermediate frequency of complementary base sequences (for example, hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less.

One skilled in the art will understand that substantially corresponding probes of the invention can vary from the referred-to sequence and still hybridize to the same target nucleic acid sequence. This variation from the nucleic acid may be stated in terms of a percentage of identical bases within the sequence or the percentage of perfectly complementary bases between the probe and its target sequence. Probes of the present invention substantially correspond to a nucleic acid sequence if these percentages are from about 100% to about 80% or from 0 base mismatches in about 10 nucleotide target sequence to about 2 bases mismatched in an about 10 nucleotide target sequence. In preferred embodiments, the percentage is from about 100% to about 85%. In more preferred embodiments, this percentage is from about 90% to about 100%; in other preferred embodiments, this percentage is from about 95% to about 100%

By “sufficiently complementary” or “substantially complementary” is meant nucleic acids having a sufficient amount of contiguous complementary nucleotides to form, under high stringency hybridization conditions, a hybrid that is stable for detection. Substantially complementary to can also refer to sequences with at least 90% identity to, e.g., 95, 96, 97, 98, 99, or 100% identity to, a given reference sequence.

The terms “identical” or percent “identity,” in the context of two or more nucleic acids or polypeptide sequences, refer to two or more sequences or subsequences that are the same or have a specified percentage of amino acid residues or nucleotides that are the same (i.e., 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site at ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1987-2005, Wiley Interscience)).

“Nucleic acid” refers to deoxyribonucleotides or ribonucleotides and polymers thereof in either single- or double-stranded form and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

By “nucleic acid hybrid” or “probe:target duplex” is meant a structure that is a double-stranded, hydrogen-bonded structure, preferably about 10 to about 100 nucleotides in length, more preferably 14 to 50 nucleotides in length, although this will depend to an extent on the overall length of the oligonucleotide probe. The structure is sufficiently stable to be detected by means such as chemiluminescent or fluorescent light detection, autoradiography, electrochemical analysis or gel electrophoresis. Such hybrids include RNA:RNA, RNA:DNA, or DNA:DNA duplex molecules.

“RNA and DNA equivalents” refer to RNA and DNA molecules having the same complementary base pair hybridization properties. RNA and DNA equivalents have different sugar groups (i.e., ribose versus deoxyribose), and may differ by the presence of uracil in RNA and thymine in DNA. The difference between RNA and DNA equivalents do not contribute to differences in substantially corresponding nucleic acid sequences because the equivalents have the same degree of complementarity to a particular sequence.

The term “isolated”, when applied to a nucleic acid or polypeptide, denotes that the nucleic acid or polypeptide is essentially free of other cellular components with which it is associated in the natural state. It can be in a homogeneous state although it can be in either a dry or aqueous solution. Homogeneity and whether a molecule is isolated can be determined using analytical chemistry techniques such as polyacrylamide gel electrophoresis or high-performance liquid chromatography. A polypeptide that is the predominant species present in a preparation is substantially isolated. The term “isolated” denotes that a nucleic acid or polypeptide gives rise to essentially one band in an electrophoretic gel. Particularly, it means that the nucleic acid or polypeptide is in some embodiments at least about 50% pure, in some embodiments at least about 85% pure, and in some embodiments at least about 90%, 95%, 96%, 97%, 98% or 99% pure.

By “preferentially hybridize” is meant that under high stringency hybridization conditions oligonucleotide probes can hybridize their target nucleic acids to form stable probe:target hybrids (thereby indicating the presence of the target nucleic acids) without forming stable probe:non-target hybrids (that would indicate the presence of non-target nucleic acids from other organisms). Thus, the probe hybridizes to target nucleic acid to a sufficiently greater extent than to non-target nucleic acid to enable one skilled in the art to accurately detect the presence of (for example Candida) and distinguish these species from other organisms. Preferential hybridization can be measured using techniques known in the art and described herein.

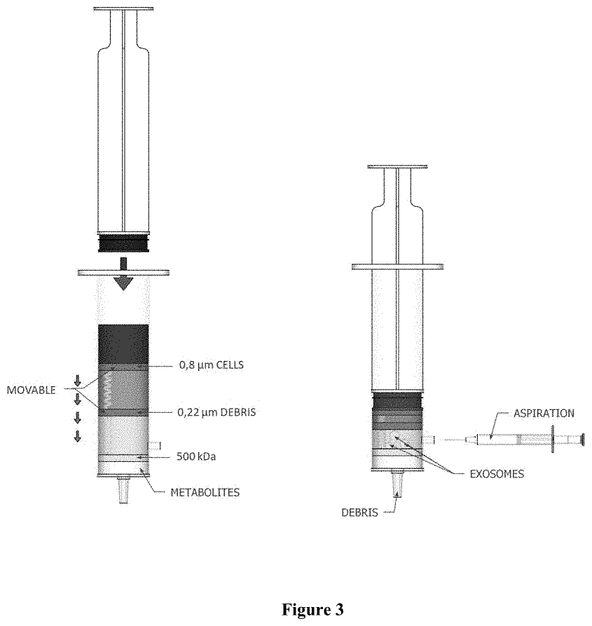

One embodiment of the invention is directed to an exosome collection kit based on sequential filtration that isolates and collects exosomes from biological fluids, such as urine and saliva. In one embodiment, the collection procedure uses a device that comprises of a 200 ml syringe with 3 integrated filters (FIG. 3). In illustrative embodiments, the invention includes a modified syringe like disposable (single use) exosome apparatus based on sequential filtration that concentrates exosomes from biological fluids, such as urine and saliva.

In one embodiment, the collection procedure uses a device that comprises of a 200 ml syringe with 3 integrated filters. The first filter is 0.01 to 1.0 micrometer filter, preferably 0.05, 0.1, 0.2, 0.3, 0.4, 0.5, 0.6, 0.7., 0.8, or 0.9 micrometer filter, with pores at the level of about 150 ml, the second filter is about 0.10 to about 0.30 micrometers, preferably 0.10. 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, or 0.30 micrometer filter, at the level of 100 ml and the third filter is a 500 kiloDalton filter, preferably 50, 100, 200, 300, 400, 500, 600, 700, 800, 900, 1000 kiloDalton filter, at the level of 50 ml.

In a preferred embodiment, the first filter is a 0.8 micrometer filter with pores at the level of 150 ml, the second filter is a 0.22 micrometer filter at the level of 100 ml and the third is a 500 kiloDalton filter at the level of 50 ml.

In certain embodiments, filters are made of materials that do not absorb proteins (PES, PVDF, regenerated cellulose). The sequential filters are connected with two appropriate springs to allow for movement of the first filter and the second micrometer filter. The springs are integrated in the hollow circumference of the syringe on either side of the filter. At the level of 50 ml, the third kiloDalton filter is immobilized. The springs give freedom of movement of the first micrometer filter and the second micrometer filter about 50 ml upwards and exactly 49 ml downwards (position 1 ml above the third kiloDalton filter). When applying maximum pressure inside the device the first micrometer filter should touch the surface of the second micrometer filter, while the latter will stop at a distance of exactly 1 ml volume between the second pressurized filter and the third kDa immobilized filter. The syringe plunger will lock in the position of maximum pressure.

The upper side of the syringe consists the biological fluid compartment of the collected samples. The lower end of the syringe can be sealed with a screw cap and will be the outlet of the treated biological fluid that does not contain exosomes (waste). The compartment between the 500 kiloDalton filter and the 0.22 micrometer filter upon maximum pressure will contain the isolated exosomes. In the side walls of the syringe there is an exit which is sealed with a screw cap. This exit will be the outlet of the material in which the exosomes are concentrated.

In one embodiment, the biological fluid sample is human urine of 50 ml volume maximum that can be immediately inserted in the upper side of the syringe. In another embodiment, biological fluid sample is human saliva that will be collected independently in a saliva collector tube and diluted to 50 ml with physiological saline and homogenized by rapid shaking before being added to the syringe.

In one embodiment, upon pressure with the syringe plunger the biological material will be pushed through the three different filters. The first micrometer filter will prevent the flow of cells and cell fragments while the second micrometer filter will prevent large vesicles, microorganisms (shed microvesicles, apoptotic particles) and protein aggregations flow. The resulting liquid is pushed through the third filter of 500 kDa, the pores of which are large enough to allow for remaining proteins and other free biomolecules to flow through. While continuous pressure is applied, exosomes are concentrated in the compartment between the second filter and the third kDa filter with a final volume of 1 ml. The springs allow the biological fluid to be pushed successively through the filters while they minimize the necessary force that must be exerted on the syringe plunger.

In one embodiment, once maximum pressure is applied and the syringe plunger is locked, waste can be disposed through the lower end of the syringe. Following re-sealing of the lower end outlet, the concentrated exosomes can be collected through aspiration with an insulin needle through the side way outlet of the exosome containing compartment.

Another aspect of the invention is directed to a diagnostic test device that simultaneously detects exosomal biomarkers based on a quantitative assay and disease (infection/inflammation) biomarkers.

In one embodiment, a rapid diagnostic device based on immunoassay tests and exosome profiling on physiological and pathological conditions will be included. The quantity of exosomes and their cargo skyrockets upon infection and disease. There is strong evidence associated with cancer, chronic diseases, inflammation, and recently it has been confirmed that viral infection has the same effect (Sur et al., 2021). The differential expression of exosomal biomarkers has shown that exosome concentration in viral infection is severity related and specific biomarkers increase in moderate and severe phenotypes compared to healthy donors (Kudryavtsev et al. 2021, Barberis et al., 2021).

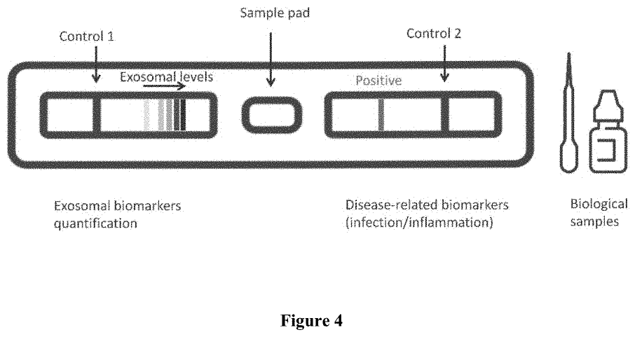

In one embodiment, the rapid diagnostic device consists of three main parts (FIG. 4). A visual read-out for exosome qualitative and quantitative identification, a visual read-out for disease (infection/inflammation) biomarkers detection, and a sample pad.

In one embodiment, the applied sample refers to the resulting liquid of concentrated exosomes collected from the exosome collection kit. In another embodiment, the applied sample is collected biological fluid (urine or saliva) from human subjects.

In one embodiment, the sample flow is directed to both ends of the device through an absorbent pad.

In one embodiment, one end of the device refers to disease (infection/inflammation) biomarkers detection and follows the principles of lateral flow immunoassay (LFIA) tests. Specific biomarkers for the disease under study are detected through the recognition of the antigens contained in the sample by a specific antibody stabilized in the strip.

The term “membrane vesicle” or “exosome” are used interchangeably and as used herein refers to essentially spherical vesicles, generally less than about 300 nm in diameter, preferably less than 250 nm, preferably less than 200 nm, preferably less than 150 nm, preferably less than 100 nm, comprising of a lipid bilayer containing a cytosolic fraction and secreted from cells. Particular membrane vesicles are more specifically produced by cells, from intracellular compartments through fusion with the plasma membrane of a cell, resulting in their release in biological fluids or in the supernatant of cells in culture. Such vesicles are generally referred to as exosomes. Exosomes can be between about 30 and about 200 nm, and more specifically between about 50 and 150 nm in diameter and, advantageously, carry membrane proteins. In addition, depending on their origin, exosomes comprise membrane proteins such as for example MHC I, MHC II, CD63, CD81 and/or HSP70 and have no endoplasmic reticulum or Golgi apparatus. Furthermore, exosomes are typically devoid of nucleic acids (e.g., DNA or RNA).

Exosome release has been demonstrated from different cell types in varied physiological contexts. For example, it has been demonstrated that B lymphocytes release exosomes carrying class II major histocompatibility complex molecules, which play a role in antigenic presentation. Similarly, it has been demonstrated that dendritic cells produce exosomes (i.e., “dexosomes” or “Dex”), with specific structural and functional characteristics and playing a role in immune response mediation, particularly in cytotoxic T lymphocyte stimulation. It has also been demonstrated that tumor cells secrete specific exosomes (i.e., “texosomes” or “Tex”) in a regulated manner, carrying tumor antigens and capable of presenting these antigens or transmitting them to antigen presenting cells (see e.g., PCT International Patent Application No. WO99/03499, herein incorporated by reference in its entirety). Also, mastocyte cells accumulate molecules in intracellular vesicular compartments, which can be secreted under the effect of signals. The kidneys also produce exosomes (i.e., urinary exosomes) (Pisitkun et al., 2004).

Therefore, as a general rule, cells appear to emit signals and communicate with each other via membrane vesicles that they release, which may carry proteins or any other signal with specific structural and functional characteristics, produced in different physiological situations. The exosome in effect is the end result of a pre-fractionation process by tissues. The vesicles are then delivered to various biological fluids, including for example blood and urine. As such, disease biology might produce a diagnostic species in increased concentration localized in membrane vesicles, including for example exosomes. Therefore membrane vesicles have value as polypeptide biomarker reservoirs and efforts to simplify the purification of membrane vesicles (e.g., exosomes) from biological fluids, including blood and urine, have diagnostic and health assessment value.

The presently disclosed subject matter provides methods of isolating membrane vesicles from biological samples. In some embodiments, the methods comprise providing a biological fluid sample comprising membrane vesicles; filtering the biological fluid sample through a exosome collection device comprising a filter having an average pore diameter of between about 0.01 μm and about 1 μm; and collecting from the exosome collection device a retentate comprising the membrane vesicles, thereby isolating the membrane vesicles from the biological fluid sample. In some embodiments, the biological sample can be treated at some point after sample collection with one or more protease inhibitors to prevent degradation of the proteins in the biological sample prior to isolation (e.g., serine protease inhibitors, chymotrypsin inhibitors, trypsin inhibitors, etc.).

The presently disclosed methods can be used to isolate membrane vesicles that maintain the presence of peripheral and integral membrane proteins, as well as globular membrane proteins. The presence of globular membrane proteins is indicative of the maintenance of the membrane vesicle structure, and little to no loss of vesicle contents.

In particular embodiments of the presently disclosed subject matter where the biological fluid is urine, the urine can be freshly collected or previously frozen urine. Additionally, the urine can be collected as a morning void/spot urine sample and/or as a mid-day void/spot urine sample. Membrane vesicles are present in urine collected at various timepoints during a day and can be isolated from both freshly collected and previously frozen urine samples. In some embodiments, the urine can also be clarified to remove, for example, casts, bacteria, and cell debris, prior to isolation of membrane vesicles by filtration. In some embodiments, the urine is clarified by low-speed centrifugation, such as for example at about 3,000 ×g, 2,000 ×g, 1,000 ×g, or less. The supernatant can then be collected, which contains the membrane vesicles, and further processed using the methods disclosed herein to isolate the exosomes.

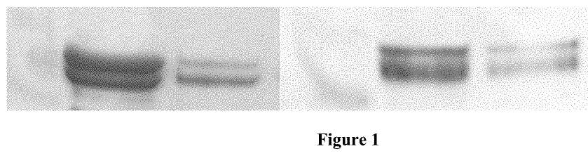

In another embodiment, the invention encompasses an exosome collection kit based on sequential filtration that isolates and collects exosomes from biological fluids, such as urine and saliva. In one embodiment, the collection procedure uses a device that comprises of a 200 ml syringe with 3 integrated filters (FIG. 1). The first filter is a 0.8 micrometer filter with pores at the level of 150 ml, the second filter is a 0.22 micrometer filter at the level of 100 ml and the third is a 500 kiloDalton filter at the level of 50 ml.

All filters are made of materials that do not absorb proteins (PES, PVDF, regenerated cellulose). The sequential filters are connected with two appropriate springs to allow for movement of, for example, the 0.8 micrometer filter and the 0.22 micrometer filter. The springs are integrated in the hollow circumference of the syringe on either side of the filter. At the level of 50 ml, the 500 kiloDalton filter is immobilized. The springs give freedom of movement of the 0.8 micrometer filter and the 0.22 micrometer filter about 50 ml upwards and exactly 49 ml downwards (position 1 ml above the 500 kiloDalton filter). When applying maximum pressure inside the device the 0.8 micrometer filter should touch the surface of 0.22 micrometer filter, while the latter will stop at a distance of exactly 1 ml volume between the 0.22 pressurized filter and the 500 kDa immobilized filter. The syringe plunger will lock in the position of maximum pressure.

In some embodiments, the exosome collection kit utilized to isolate the membrane vesicles from the biological sample is a fiber-based filtration cartridge. In some embodiments, the fibers are hollow polymeric fibers, such as for example polypropylene hollow fibers. In these embodiments, sample can be introduced into the exosome collection kit by pumping the sample fluids into the module with a pump device, such as for example a peristaltic pump. The pump flow rate can vary, but in some embodiments, the pump flow rate is set at about 2 mL/minute.

In some embodiments, the exosome collection kit utilized to isolate the membrane vesicles from the biological sample is a membrane exosome collection device. For example, in some embodiments, the membrane exosome collection kit comprises a filter disc membrane (e.g., a hydrophilic polyvinylidene difluoride (PVDF) filter disc membrane) housed in a stirred cell apparatus (e.g., comprising a magnetic stirrer). In some embodiments, the sample moves through the filter as a result of a pressure gradient established on either side of the filter membrane.

In some embodiments, the filter within the exosome collection kit that retains the membrane vesicles (i.e., the retentate) from the biological fluid sample (i.e., the filtrate) has an average pore diameter sufficient for exosome retention and permeation of all but the largest proteins. For example, in some embodiments, the filter has an average pore diameter of about 0.01 μm to about 0.15 μm, and in some embodiments from about 0.05 μm to about 0.12 μm. In some embodiments, each of the filters has an average pore diameter of about 0.01 μm, 0.02 μm, 0.03 μm, 0.04 μm, 0.05 μm, 0.06 μm, 0.07 μm, 0.08 682 m, 0.09 μm, 0.1 μm, 0.2 μm, 0.3 μm, 0.4 μm, 0.5 μm, 0.6 μm, 0.7 μm, 0.8 μm, 0.9 μm, or 1.0 μm. In some embodiments, the filter utilized comprises a material having low hydrophobic absorptivity and/or high hydrophilic properties. In particular embodiments, the filter has an average pore size for exosome retention and permeation of most proteins as well as a surface that is hydrophilic, thereby limiting protein adsorption. Similar filters with these properties can also be suitably used with the presently disclosed subject matter. For example, in some embodiments, the filter comprises a material selected from the group consisting of polypropylene, PVDF, polyethylene, polyfluoroethylene, cellulose, secondary cellulose acetate, polyvinylalcohol, and ethylenevinyl alcohol (Kuraray Co., Okayama, Japan). Additional materials that can be utilized in filters of certain embodiments include, but are not limited to, polysulfone and polyethersulfone.

The retentate comprising the isolated membrane vesicles is collected from the exosome collection device. In some embodiments, the retentate is collected by flushing the retentate from the filter. Selection of a filter composition having hydrophilic surface properties, thereby limiting protein adsorption, can facilitate easier collection of the retentate and minimize use of harsh or time-consuming collection techniques. Once collected the membrane vesicles and/or associated polypeptide biomarkers can be further purified and/or concentrated and finally suspended in a suitable buffer solution, such as for example phosphate buffered saline (PBS), depending on how the vesicles and/or polypeptides will be utilized.

The upper side of the syringe consists the biological fluid compartment of the collected samples. The lower end of the syringe can be sealed with a screw cap and will be the outlet of the treated biological fluid that does not contain exosomes (waste). The compartment between the kiloDalton filter and the second micrometer filter upon maximum pressure will contain the isolated exosomes. In the side walls of the syringe there is an exit which is sealed with a screw cap. This exit will be the outlet of the material in which the exosomes are concentrated.

In one embodiment, the biological fluid sample is human urine of 50 ml volume maximum that can be immediately inserted in the upper side of the syringe. In another embodiment, biological fluid sample is human saliva that will be collected independently in a saliva collector tube and diluted to 50 ml with physiological saline and homogenized by rapid shaking before being added to the syringe.

In one embodiment, upon pressure with the syringe plunger the biological material will be pushed through the three different filters. The first 0.8 micrometer filter will prevent the flow of cells and cell fragments while the second 0.22 micrometer filter will prevent large vesicles, microorganisms (shed microvesicles, apoptotic particles) and protein aggregations flow. The resulting liquid is pushed through the final filter of 500 kDa, the pores of which are large enough to allow for remaining proteins and other free biomolecules to flow through. While continuous pressure is applied, exosomes are concentrated in the compartment between the second 0.22 micrometer filter and the third 500 kDa filter with a final volume of 1 ml. The springs are essential so that the biological fluid is pushed successively through the filters while they minimize the necessary force that must be exerted on the syringe plunger.

In one embodiment, once maximum pressure is applied and the syringe plunger is locked, waste can be disposed through the lower end of the syringe. Following re-sealing of the lower end outlet, the concentrated exosomes can be collected through aspiration with an insulin needle through the side way outlet of the exosome containing compartment.

Once isolated, the membrane vesicles can be analyzed to identify characteristics of the vesicles, including identification and/or quantitation of exosomal polypeptides.

Identification and/or quantitation of polypeptides within the vesicle can provide information related to biomarkers expressed within a subject. The identification of biomarkers expressed in a subject can be utilized to diagnose a disorder in a subject, monitor the progress of treatment of a disorder in a subject, and generally determine the state of health of a subject as a baseline, or as compared to a previously determined biomarker analysis.

As such, the presently disclosed subject matter provides methods of identifying and/or quantitating biomarker polypeptides from a biological fluid sample using the membrane vesicle isolation methods disclosed herein. The isolated membrane vesicles can then be subjected to polypeptide separation and/or analysis procedures generally known in the art to identify and quantitate the biomarker polypeptides associated with the isolated vesicles.

The invention further provides methods of diagnosing a disorder or measuring a disorder state in a subject utilizing the membrane vesicle isolation techniques disclosed herein in combination with polypeptide isolation and quantitation techniques. For example, water channel aquaporin 2 (AQP2) is a biomarker for certain water-balance disorders and identification of peptide variants expressed by a subject can provide information related to diagnosis of the disorders. Other non-limiting examples of disorders that can be diagnosed and/or monitored based on biomarker identification and/or quantitation include, but are not limited to diabetes, myocardial ischemia (troponin); cardiovascular risk (C-reactive protein, homocysteine); prostatic hypertrophy and prostatic cancer (PSA); systemic lupus erythematosus (ANA); and rheumatoid arthritis (Rheumatoid factor), with non-limiting exemplary biomarkers listed in parenthesis.

Table 1 provides a non-limiting, illustrative, exemplary list of exosomal biomarkers with sensitivity upon infection/inflammation

TABLE 1

| Exosomal Proteins | Pathology |
|---|---|
| CD9 | Covid-19, Renal Cell Carcinoma, Diabetes |
| CD81 | Covid-19, Hepatitis C, Cancer |
| CD63 | Covid-19, Melanoma, Prostate cancer, Breast |
| | cancer |
| CD147 | Covid-19, Cancer |
| CD235 | Covid-19, HIV1, Dengue virus, Amyotrophic |
| | Lateral Sclerosis |
| CD41 | Covid-19, Inflammation, Ovarian cancer, |
| | Eosinophilic esophagitis |
| COPB2 (coat complex | Covid-19, Hepatocellular carcinoma, Prostate |
| subunit beta 2) | cancer, Glioma |
| PRKCB (protein kinase | Covid-19, Prostate cancer, Breast cancer, |
| C beta) | schizophrenia |
| RHOC (ras homolog | Covid-19, Cancer |
| family member C) | |
| KRAS (KRAS proto- | Covid-19, Cancer |
| oncogene) | |
| CRP (C-reactive | Covid-19, Inflammation, Dengue virus |
| protein) | |
| CAPN2 (calpain 2) | Covid-19, HBV, Prostate cancer, Renal cell |
| | carcinoma |
| ECM1 (extracellular | Covid-19, Cancer, Azoospermia |
| matrix protein 1) | |
| FGG (fibrinogen | Covid-19, Cancer, Chronic obstructive |
| gamma chain) | pulmonary disease (COPD) |
| MFAP4 (microfibril- | Covid-19, Chronic obstructive pulmonary |
| associated glycoprotein | disease (COPD), Hepatitis C |
| 4 precursor) | |
| Exosomal RNAs | Pathology |
| miR-15 | Covid-19, Cataract, Diabetes, Cancer |
| miR-24-3p | Covid-19, Depression, Cancer |
| hsa-miR-203-3p | Covid-19, Viral replication, Cancer |
| hsa-miR-4482-3p | Covid-19, Viral replication |
| hsa-miR-44366b-3p | Covid-19, Viral replication |
| hsa-miR-190a-5p | Covid-19, Chronic obstructive pulmonary |
| | disease (COPD), Immune suppression |
| miR-122-5p | Covid-19, Acute myocardial infarction, Cancer |
| SNORD33 | Covid-19, Cancer |
| AL732437.2 | Covid-19 |
| RNU2-29P | Covid-19 |
| CDKN2B-AS1 | Covid-19, Diabetic nephropathy (DN), Canceer |
| AL365184.1 | Covid-19 |
| Exosomal lncRNAs | Pathology |
| LINC00657 | Ovarian Cancer |
| LRRC75A-AS1 | Colorectal Cancer |
| CRNDE | Colorectal Cancer |
| SNHG16 | Cancer |
| ZFAS1 | Colorectal Cancer |
| MALAT-1 | Non-Small Cell Lung Cancer |

Further with respect to the diagnostic methods of the presently disclosed subject matter, a preferred subject is a vertebrate subject. A preferred vertebrate is warm-blooded; a preferred warm-blooded vertebrate is a mammal. A preferred mammal is most preferably a human. As used herein, the term “subject” includes both human and animal subjects. Thus, veterinary therapeutic uses are provided in accordance with the presently disclosed subject matter.

As such, the presently disclosed subject matter provides for the diagnosis of mammals such as humans, as well as those mammals of economic importance, such as animals raised on farms for consumption by humans; and/or animals of social importance to humans, such as animals kept as pets or in zoos. Examples of such animals include but are not limited to carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses, poultry, and the like.

As disclosed, polypeptides from the isolated membrane vesicles can be separated and analyzed to identify and/or quantitate the polypeptides. Polypeptide separation techniques are generally known in the art and include, for example, electrophoretic and/or chromatographic techniques (e.g., liquid chromatography) and immunoisolation. Polypeptide identification and quantitation techniques are also well-known in the art.

Numerous methods and devices are well known to the skilled artisan for the detection and analysis of polypeptides, which are applicable to detection and analysis of isolated biomarker peptides associated with isolated exosomes. For example, mass spectrometry and/or immunoassay devices and methods can be used, although other methods are well-known to those skilled in the art. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety. These devices and methods can utilize labeled molecules in various sandwich, competitive, or non-competitive assay formats, to generate a signal that is related to the presence and/or amount of a biomarker polypeptide of interest. Additionally, certain methods and devices, such as biosensors and optical immunoassays, can be employed to determine the presence or amount of analytes without the need for a labeled molecule. See, e.g., U.S. Pat. Nos. 5,631,171; and 5,955,377, each of which is hereby incorporated by reference in its entirety.

In certain embodiments of the presently disclosed subject matter, the biomarker peptides are analyzed using an immunoassay. The presence or amount of a biomarker peptide can be determined using antibodies or fragments thereof specific for each marker and detecting specific binding. For example, in some embodiments, the antibody specifically binds a polypeptide of Table 1. In some embodiments, the antibody is a monoclonal antibody. Any suitable immunoassay can be utilized, for example, Western blots, enzyme-linked immunoassays (ELISA), radioimmunoassays (RIAs), competitive binding assays, and the like. Specific immunological binding of the antibody to the marker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The use of immobilized antibodies or fragments thereof specific for the markers is also contemplated by the present subject matter. The antibodies can be immobilized onto a variety of solid supports, such as magnetic or chromatographic matrix particles, the surface of an assay plate (such as microtiter wells), pieces of a solid substrate material (such as plastic, nylon, paper), and the like. An assay strip can be prepared by coating the antibody or a plurality of antibodies in an array on solid support. This strip can then be dipped into the test biological sample and then processed quickly through washes and detection steps to generate a measurable signal, such as for example a colored spot.

The analysis of a plurality of markers is contemplated by the presently disclosed subject matter and can be carried out separately or simultaneously with one or more test samples.

In certain embodiments, several markers can be combined into one test for efficient processing of a multiple of samples. In addition, one skilled in the art would recognize the value of testing multiple samples (for example, at successive time points) from the same subject. Such testing of serial samples provides for the identification of changes in biomarker polypeptide levels over time. Increases or decreases in marker levels, as well as the absence of change in marker levels, can provide useful information about the disease status that includes, but is not limited to identifying the approximate time from onset of the event, the presence and amount of salvageable tissue, the appropriateness of drug therapies, the effectiveness of various therapies as indicated by reperfusion or resolution of symptoms, differentiation of the various types of a disorder, identification of the severity of the event, identification of the disease severity, and identification of the subject's outcome, including risk of future events.

A panel consisting of biomarkers associated with a disorder can be constructed to provide relevant information related to the diagnosis or prognosis of the disorder and management of subjects with the disorder. Such a panel can be constructed, for example, using 1, 2, 3, 4, 5, 6, 7, 8, 9, 10, 15 or 20 individual biomarkers. The analysis of a single marker or subsets of markers comprising a larger panel of markers could be carried out by one skilled in the art to optimize clinical sensitivity or specificity in various clinical settings. These include, but are not limited to ambulatory, urgent care, critical care, intensive care, monitoring unit, in subject, out subject, physician office, medical clinic, and health screening settings. The analysis of biomarker polypeptides could be carried out in a variety of physical formats as well. For example, the use of microtiter plates or automation could be used to facilitate the processing of large numbers of test samples. Alternatively, single sample formats could be developed to facilitate immediate treatment and diagnosis in a timely fashion, for example, in ambulatory transport or emergency room settings.

In some embodiments, a kit for the isolation and analysis of biomarker polypeptides is provided that comprises a exosome collection device comprising a filter having an average pore diameter of between about 0.01 μm and about 1.0 μm and antibodies or fragments thereof having specificity for one or more biomarker polypeptides of interest. Such a kit can comprise devices and reagents for the analysis of at least one test sample. The kit can further comprise instructions for using the kit and conducting the analysis. Optionally the kit can contain one or more reagents or devices for converting a marker level to a diagnosis or prognosis of the subject.

Further, mass spectrometry is a useful and well-characterized tool for polypeptide identification and quantitation, alone or in combination with polypeptide separation techniques, particularly when coupled with bioinformatics analysis. Peptide molecular weights and the masses of sequencing ions can be obtained routinely using mass spectrometry to an accuracy which enables mass distinction amongst most of the 20 amino acids in the genetic code, as well as quantitation of peptides in a sample. Single or tandem mass spectrometry can be used. In tandem mass spectrometry, a peptide sample is introduced into the mass spectrometer and is subjected to analysis in two mass analyzers (denoted as MS1 and MS2). In MS1, a narrow mass-to-charge window (typically 2-4 Da), centered around the m/z ratio of the peptide to be analyzed, is selected. The ions within the selected mass window are then subjected to fragmentation via collision-induced dissociation, which typically occurs in a collision cell by applying a voltage to the cell and introducing a gas to promote fragmentation. The process produces smaller peptide fragments derived from the precursor ion (termed the ‘product’ or daughter ions). The product ions, in addition to any remaining intact precursor ions, are then passed through to a second mass spectrometer (MS2) and detected to produce a fragmentation or tandem (MS/MS) spectrum. The MS/MS spectrum records the m/z values and the instrument-dependent detector response for all ions exiting from the collision cell. Fragmentation across the chemical bonds of the peptide backbone produces ions that are either charged on the C-terminal fragment (designated as x, y or z ions) or on the N-terminal fragment (a, b or c ions). Peptides are fragmented using two general approaches, high and low energy collision-induced dissociation (CID) conditions. In low energy CID experiments, signals assigned to y and b ions and from losses of water and ammonia are usually the most intense. During high energy CID, peptide molecules with sufficient internal energy to cause cleavages of the amino acid side chains are produced. These side chain losses predominantly occur at the amino acid residue where the backbone cleavage occurs. The general designations for these ions are d for N-terminal and w for C-terminal charged fragments, respectively. Other useful sequencing ions occur which result from a y-type cleavage at one residue and a b type cleavage at another residue along the polypeptide backbone (internal fragment ions) (Biemann, 1990; Papayannopoulos, 1995).

In one embodiment, the polypeptides are separated and analyzed using matrix-assisted laser-desorption time-of-flight mass spectrometry (MALDI-TOF). This instrument configuration is used to generate a primary mass spectrum in order to determine the molecular weight of the polypeptide. Other mass spectrometric techniques include, without limitation, time-of-flight, Fourier transform ion cyclotron resonance, quadrupole, ion trap, and magnetic sector mass spectrometry and compatible combinations thereof. See for example U.S. Pat. Nos. 6,925,389; 6,989,100; and 6,890,763 for further guidance, each of which is incorporated by reference herein in its entirety.

With regard to proteomic analysis, various computer-mediated methods are known for deducing the sequence of a peptide from an MS/MS spectrum. In one approach, sub-sequencing' strategies are used whereby portions of the total sequence, (i.e., sub-sequences) are tested against the mass spectrum (see Ishikawa et al., 1986; Siegel et al., 1988; Johnson et al., 1989, each of which is hereby incorporated by reference in its entirety). In this approach, sub-sequences that read or correlate to ions observed in the MS/MS spectrum are extended by a residue and the whole process is then repeated until the entire sequence is obtained. During each incremental extension of the sequence, the possibilities are reduced by comparing sub-sequences with the mass spectrum and only permitting continuation of the process for sub-sequences giving the most favorable spectral matches. Determination of amino acid composition has also been utilized to limit sequence possibilities (Zidarov et al., 1990, hereby incorporated by reference in its entirety).

Another approach utilizes computer programs for de novo peptide sequencing from fragmentation spectra based on graph theory (Fernandez-de-Cossio et al., 1995; Hines et al., 1995; Knapp, 1995, which are hereby incorporated by reference in their entirety). The basic method involves mathematically transforming an MS/MS spectrum into a form where fragment ions are converted to a single fragment ion type represented by a vertex on the spectrum graph (Bartels, 1990, the contents of which is hereby incorporated by reference in its entirety). Peptide sequences are then determined by finding the longest series of these transformed ions with mass differences corresponding to the mass of an amino acid.

Other methods match spectral information with sequences in protein and translated nucleotide sequence databases. An algorithm has been described for searching protein and nucleotide databases with mass and sequence information from fragmentation spectra of tryptic peptides (MS-TAG) (Mann and Wilm, 1994; Clauser et al., 1996, which are hereby incorporated by reference in their entirety). A comparison with the fragmentation spectra of the same peptide after methylation of the carboxyl groups or enzymatic digestion in the presence of 180 water to incorporate 180 into the C-terminal carboxy groups (Shevchenko et al., 1997, which is hereby incorporated by reference in its entirety) can provide even more accurate results. A similar approach has been extended to the analysis of intact proteins using laser fragmentation and Fourier-transform mass spectrometry (Mortz, E. et al., 1996, which is hereby incorporated by reference in its entirety).

Another approach has been described for identifying peptide sequences from database interrogation by comparing the experimental fragmentation spectrum with theoretical spectra from a mass-constrained set of database sequences (SEQUEST) (U.S. Pat. No. 5,538,897; Yates et al., 1991, which are hereby incorporated by reference in their entirety). For each candidate sequence within the database spectrum, a theoretical fragmentation spectrum is formed according to a selected ion model of peptide fragmentation. The predicted theoretically derived mass spectra are compared to each of the experimentally derived fragmentation spectra by a cross-correlation function for scoring spectra.

Another aspect of the invention is directed to a diagnostic test device that simultaneously detects exosomal biomarkers based on a quantitative assay and disease (infection/inflammation) biomarkers.

In one embodiment, a rapid diagnostic device based on immunoassay tests and exosome profiling on physiological and pathological conditions will be included. The quantity of exosomes and their cargo skyrockets upon infection and disease. There is strong evidence associated with cancer, chronic diseases, inflammation, and recently it has been confirmed that viral infection has the same effect (Sur et al., 2021). The differential expression of exosomal biomarkers has shown that exosome concentration in viral infection is severity related and specific biomarkers increase in moderate and severe phenotypes compared to healthy donors (Kudryavtsev et al. 2021, Barberis et al., 2021).

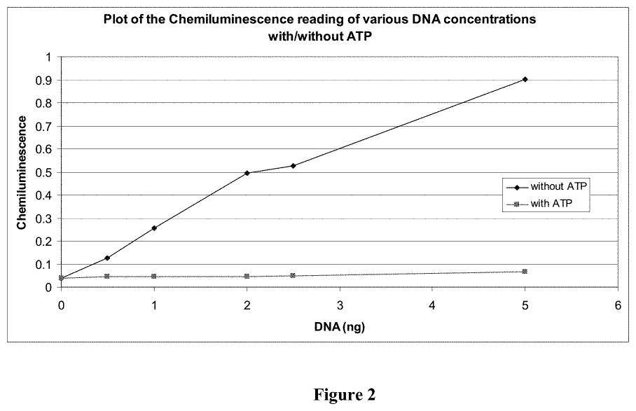

In one embodiment, the rapid diagnostic device consists of three main parts (FIG. 2). A visual read-out for exosome qualitative and quantitative identification, a visual read-out for disease (infection/inflammation) biomarkers detection, and a sample pad.

In one embodiment, the applied sample refers to the resulting liquid of concentrated exosomes collected from the exosome collection kit. In another embodiment, the applied sample is collected biological fluid (urine or saliva) from human subjects.

In one embodiment, the sample flow is directed to both ends of the device through an absorbent pad.

In one embodiment, one end of the device refers to disease (infection/inflammation) biomarkers detection and follows the principles of lateral flow immunoassay (LFIA) tests. Specific biomarkers for the disease under study are detected through the recognition of the antigens contained in the sample by a specific antibody stabilized in the strip.

In one embodiment, specific biomarkers for exosomal biomarkers upon, for example, inflammation and infection have been recognized. As listed in Table 1, exosomal proteins and exosomal RNAs are defined by the inventors, as a collection of exosomal biomarkers with predictive value for disease severity and clinical manifestation of infection/inflammation.

In one embodiment, the other end of the device refers to exosome identification and quantification based on a quantitative lateral flow assay (LFA). Specific exosome biomarkers related to the disease under study are detected through the recognition of the antigens (exosomal biomarkers) contained in the sample by a specific antibody stabilized in the strip. The visual read-out is accordingly adjusted so that the ink advancement distance will be proportional to the levels of detected exosomes from the sample.

One aspect of the invention is a rapid, non-invasive detection system for diagnostics with higher accuracy. The diagnostic test device quantifies the levels of disease-related exosomes and determines the severity and/or progression of the disease. In one embodiment, the diagnostic test device minimizes false positive and false negative results by employing the sensitivity of disease-related exosomes and the simultaneous detection of disease-specific biomarkers. Another aspect of the invention is the application of the detection test device for prognosis of disease progression based on the quantification of disease-related exosomal levels.

### EXAMPLES

For the selection of compound datasets and their high-throughput virtual screening, a series on in-house kept compound databases were used. Those databases have been based on both computational and experimental criteria.

The experimental techniques that have been used revolve around analog assays to a previously custom-designed enzymatic assay for the rapid screening of anti-viral helicase modulators. The techniques and methodologies used are described below:

Prepare all solutions using ultrapure water, prepared by purifying deionized water and store all reagents at room temperature, unless indicated otherwise.

1.1 HCV Recombinant Helicase Preparation

1. Escherichia coli induction: Prepare 4 conical flasks with 750 mL of LB and add 34 μg/mL chloramphenicol and 25 μg/mL kanamycin to each.

2. Cell lysis buffer: 20 mM sodium phosphate pH 7.5, 300 mM NaCl

3. Buffer S: 20 mM sodium phosphate pH 7.4, 500 mM NaCl

4. Exchange buffer: 25 mM Tris-HCl pH 7.5, 0.05% CHAPS (3-[(3-cholamidopropyl)-dimethylammonio]-1-propane sulphonate), 20% glycerol, 5 mM DTT (dithiothreitol)

1.2 HCV Helicase Assay Procedure

1. Oligonucleotide mix:

Oligonucleotides (5′-biotinGCTGACCCTGCTCCCAATCGTAATCTATAGTGTCACCTA-3′, 5′-DIG-CGATTGGGAGCAGGGTCAGC-3′) 1:1 molar, HEPES 2 mM, NaCl 0.05M, EDTA 0.1 mM, SDS 0.01% w/v.

2. Neutravidin stock solution: add neutravidin in final concentration 1 mg/ml in phosphate buffered saline (1 M PBS—pH 7.0).

3. BSA solution: 0.1% w/v BSA.

4 Substrate solution: mix 2.5 ng partially annealed DNA duplex and 75 μL 1 M PBS containing 1M NaCl, for each well.

5. Substrate wash solution: 50 mM Tris HCl pH 7.5, 50 mM NaCl.

6. Helicase reaction mix: 11 nM purified full-length HCV NS3 protein, 25 mM 4-morpholine-propanesulphonic acid (MOPS) pH 7.0, 2 mM DTT, 3 mM MnCl2, 100 μg/ml of BSA and 5 mM ATP. The reaction mix for the negative control lacks ATP.

7. Reaction wash solution: 150 mM NaCl

8. Detection washing buffer: 0.1 M maleic acid, 0.15 M NaCl, 0.3%, Tween20, pH 7.5

9. Blocking solution: 10% BSA w/v, 0.1 M maleic acid, 0.15 M NaCl, pH 7.5

10. Antibody solution: 1:10.000 solution of the anti-Dig antibody (75 mU/mL) in Blocking solution

11. Detection buffer: 0.1 M Tris-HCl, 0.1 M NaCl, pH 9.5

12. Chemiluminescence substrate solution: CSPD—0.25 Mm

All Procedures are Carried out at Room Temperature, unless Specified Otherwise

2.1 HCV Recombinant Helicase NS3 Preparation

1. Insert the full-length HCV helicase coding region in a pET28a vector, with a N-terminal 6xHis-Tag region.

2. Verify the intactness of the gene before inducing the protein production.

3. Transform Escherichia coli cells (strain C41, DE3) with the helicase plasmid and1 inoculate the prepared LB flasks with them.

4. Induce the recombinant helicase production, adding 0.5 mM IPTG to each flask and then allow the cultures to grow for 3 hours at 18° C.

5.Resuspend the cell pellet from the 4 cultures in 30 mL Lysis buffer and then add lysozyme 100 μg/mL and Triton X-100 0.1%.

6. Incubate on ice for 30 min and then sonicate four times for 20 sec with 15 sec intervals.

7. Centrifuge the suspension at 15.000 ×g for 20 min.

8. Adjust the clarified homogenates to 10 mM imidazole and filter them through a 0.45 μm membrane.

9. Load the homogenates twice on nickel affinity columns (Ni-NTA).

10. Wash each column with five times the column volume of buffer S, containing 10 mM imidazole.

11. Elute the NS3 helicase with buffer S containing 300 mM imidazole.

12. Immediately after the elution, exchange the buffer in the helicase-containing fractions for the Exchange buffer, via dialysis. This step is critical in order to avoid precipitation.

13. Evaluate the protein concentration using the Bradford assay with BSA as standard.

14. Create aliquots of the NS3 helicase and store at −80° C.

This recombinant protein preparation is estimated to be more than 85% pure by SDS gel electrophoresis and Coomasie blue staining, yielding almost 1.6 mg of HCV NS3 per 3 litres of E. coli cultures (FIG. 1).

Chemiluminesence readings were taken using all different control combinations (presence/absence of helicase, DNA substrate and ATP) of the experiment in order to ensure the reliability of the measurements (Table 1).

TABLE 1
Each enzymatic activity assay was performed in triplicate and the results
were averaged. All reactions were allowed to proceed for 60 minutes
(concentrations as described in the methods section).

| | CHEMILUMINESENCE |
|---|---|
| +DNA substrate − helicase − ATP | 0.532 (±2%) |
| +DNA substrate − helicase + ATP | 0.529 (±4%) |
| +DNA substrate + helicase − ATP | 0.525 (±5%) |
| −DNA substrate + helicase + ATP | 0.040 (±3%) |

We demonstrated that the NS3-mediated unwinding is proportional to the amount of DNA substrate in the well, but also to the HCV helicase concentration in the reaction. The reactions were ATP-dependent (Table 2 and FIG. 2).

TABLE 2
Different DNA substrate concentrations (60-minute run).

| DNA (ng) | | |
|---|---|---|
| immobilized | CHEMILUMINESENCE | CHEMILUMINESENCE |
| per well | without ATP | with ATP |
| 0 | 0.039 | 0.040 |
| 0.5 | 0.128 | 0.044 |
| 1 | 0.255 | 0.045 |
| 2 | 0.495 | 0.045 |
| 2.5 | 0.525 | 0.048 |
| 5 | 0.902 | 0.065 |

2.2 Annealing Reaction

1. Prepare for annealing by heating the oligonucleotide mix at 100° C. for 5 min.

2. Incubate at 65° C. for 30 min and then at 22° C. for 4 h, to allow gradual annealing.

3. Store the annealed NS3 helicase substrate at −20° C. (See Note 1).

2.3 Neutravidin Coating of the 96-Well Plates

1. Coat each of the 96 wells overnight at 4° C. with 100 μl/well of a 5 μg/ml neutravidin solution in 0.5 M sodium carbonate buffer pH 9.3.

2. Wash the plates three times with 100 μl/well of PBS and air-dry at room temperature.

2.4 Blocking with BSA

1. Add 100 μL of the 0.1% w/v BSA solution

2. Incubate at 22° C. for 2 h.

3. Wash the plate three times with PBS, 200 μl/well and air-dry at room temperature.

4. Store the plate at 4° C. with desiccant (See Note 2)

2.5 Substrate Application in the 96-Well Plate

1. Pre-warm all solutions to 37° C. (See Note 3)

2. Mix 75 μl of the Substrate solution with 2.5 ng of the partially annealed DNA duplex for each well.

2. Incubate at 22° C. for 4 h.

3. Wash each well twice with 200 μl PBS per well and once with Substrate Wash solution.

2.6 Helicase Reaction

1. Add 90 μL of the Reaction mix per well.

2. Incubate the reactions at 37° C. for 1 hour.

3. Wash the plate twice with 200 μL Reaction Washing Buffer per well and let dry for 15 minutes at room temperature.

2.7 Activity Determination—Chemiluminescence Preparation

1. Wash all wells for 5 min with the Detection washing buffer.

2. Then fill up each well with Blocking solution for 30 min and then incubate for 30 min in 20 μl Antibody solution.

3. Wash twice the plate with 100 μl of Detection buffer.

4. Apply 20 μL of Detection buffer for equilibration and 1 μL of chemiluminescence substrate working solution per well and incubate for 5 min at 17° C.

5. Drain the wells and incubate the plate at 37° C. for 30 min to allow any remaining solution to evaporate.

6. The luminescence has a constant intensity for about 24 hours and continues for approximately 48 hours. The remaining DIG in each well is counted for 10 min against controls (one of which lacks protein and the other lacks ATP) in a luminescence plate reader (See Note 4).

Note 1: The oligonucleotides used in this protocol are the ones described by Hicham Alaoui-Ismaili et al. (Hicham et al., 2000), modified by DIG labelling of the release strand. Other sequences could work, but a 3′ single stranded region in the substrate is necessary to initiate the strand displacement (Tai et al., 1996).

Note 2: As far as the blocking procedure is concerned, it is important to ensure that all potential binding sites are occupied, to prevent direct binding of the detection antibody to the well in a later step. The wells should be filled with blocking solution, fully coating the plate.

Note 3: Pre-warming the solutions allows reactions to proceed at their optimum temperatures and avoids rate changes due to temperature equilibration.

Note 4: All the reaction wells should be filled up with blocking solution, to ensure that the whole well has been blocked, preventing non-specific binding of any components of the detection system.

3. Generic Molecular Biology Techniques Used

3.1 Bacterial Transformation:

1. 1 μL of plasmid DNA added to cells

2. Leave on ice for 45 minutes

3. Heat in waterbath at 42 ° C. for 2 minutes

Add 0.5 μL of LB medium with no antibiotic

Leave on ice for 1 hour at 37 ° C.

Microcentrifuge at 8000 rpm for 1 minute

Take off and discard 0.5 mL of supernatant

Re-suspend gently in remaining volume

Plate on LB agar+AMP Petri dishes

3.2 E. coli Culture

Pick up a healthy looking (round, consistent) colony with a loop

Inoculate in a universal tube with 10 mL of LB+AMP

Incubate in shaking centrifuge at 37° C. for 13 to 14 hours

3.3 Mini-Prep Preparation

1. Add 1.5 mL of overnight culture of E.Coli in an eppendorf and spin at 13000 for 1 minute

2. Repeat 3 times for each tube→passing a total of 4.5 mL of cell suspension from each tube

Each Time Discard Supernatant

3.4 Mini—Prep

using a microcentrifuge

This protocol is designed for purification of up to 20 μg of high-copy plasmid DNA from 1-5 mL overnight cultures of E. coil in LB (Luria-Bertani) medium.

Note: All protocol steps should be carried out at room temperature.

Procedure

1. Re-suspend pelleted bacterial cells in 250 μL Buffer P1 and transfer to a microcentrifuge tube.

2. Ensure that RNase A has been added to Buffer P1 No cell clumps should be visible after resuspension of the pellet.

3. Add 250 μL Buffer P2 and gently invert the tube 4-6 times to mix.

4. Mix gently by inverting the tube. Do not vortex, as this will result in shearing of genomic DNA. If necessary, continue inverting the tube until the solution becomes viscous and slightly clear. Do not allow the lysis reaction to proceed for more than 5 min.

5. Add 350 μL Buffer N3 and invert the tube immediately but gently 4-6 times.

6. To avoid localized precipitation, mix the solution gently but thoroughly, immediately after addition of Buffer N3. The solution should become cloudy.

7. Centrifuge for 10 min at 13.000 rpm (˜17.900 ×g) in a table-top microcentrifuge.

8. A compact white pellet will form.

9. Apply the supernatants from step 4 to the QIAprep Spin Column by decanting or pipetting.

10. Centrifuge for 30-60 s. Discard the flow-through.

11. (Optional): Wash the QIAprep Spin Column by adding 0.5 mL Buffer PB and centrifuging for 30-60 s. Discard the flow-through.

12. This step is necessary to remove trace nuclease activity when using endA+ strains such as the JM series, HB101 and its derivatives, or any wild-type strain, which have high levels of nuclease activity or high carbohydrate content. Host strains such as XL-1 Blue and DH5α™ do not require this additional wash step.

13. Wash QIAprep Spin Column by adding 0.75 mL Buffer PE and centrifuging for 30-60 s.

14. Discard the flow-through, and centrifuge for an additional 1 min to remove residual wash buffer

15. Note: Residual wash buffer will not be completely removed unless the flow-through is discarded before this additional centrifugation. Residual ethanol from Buffer PE may inhibit subsequent enzymatic reactions.

16. Place the QIAprep column in a clean 1.5 mL microcentrifuge tube. To elute DNA, add 50 μL Buffer EB (10 mM Tris.Cl,pH 8.5) or water to the center of each QIAprep

17. Spin Column, let stand for 1 min, and centrifuge for 1 min.

3.5 DNA Concentration Protocol

Materials:

1. TE solution

10 mM Tris (pH to 7.5)

1 mM EDTA (pH to 8.0 to dissolve)

2. DNA sample

3. SYBR Green I(R) nucleic acid gel stain (Molecular Probes)

4. Plastic wrap

5. distilled water

6. DNA marker stock (10 mg/mL)

Supplies:

1. Tubes

2. Polaroid setup (with proper filter—SYBR Green/Gold gel stain photographic filter) and UV light box

3. Micropipetter and tips

Procedures:

1. Prepare 6 DNA standards from DNA marker stock:

standard I (5 ug/ul): 1:2 dilution of DNA marker stock

standard II (2.5 ug/ul): 1:2 dilution of standard I

standard III (1.25 ug/ul): 1:2 dilution of standard II

standard IV (0.625 ug/ul): 1:2 dilution of standard III

standard V (0.313 ug/ul): 1:2 dilution of standard IV

standard VI (0.156 ug/ul): 1:2 dilution of standard V

1. Make a 1:5000 dilution of the SYBR Green I(R) with TE solution.

2. Mix 5 ul of the DNA sample and each of the 6 standards with 5 ul of the diluted SYBR Green I(R) dye.

3. Place a sheet of plastic wrap smoothly onto the UV light box.

4. Spot the mixtures individually onto plastic wrap.

5. Spot the set of 6 standards.

6. Turn on the UV light and take a photo (Polaroid 667 black-and-white print film).

7. Compare the brightness of the DNA sample with the DNA standards and estimate concentration.

3.6 Ligation

Ligation—PCR

Add 1 μL of the vector

Add 1 μL of 10× buffer

Add PCR product (1 μL)

Add 8 μL of SIGMA water

Add 1 μL of Ligase

Ligation—Overnight

Add 0.5 μL of the vector

Add 5 μL of 10× buffer

Add 3.5 μL Insert DNA

Add 1 μL of Ligase (last)

Incubate overnight at 17 ° C.

Ligation—Benchtop

Add 0.5 μL of the vector

Add 5 μL of 10× buffer

Add 3.5 μL Insert DNA

Add 1 μL of Ligase (last)

Leave at room temperature for 2 hours +30 minutes.

3.7 SDS-Page Gel Preparation

| | 10% Running Gel | Stacking Solution |
|---|---|---|
| Acrylamide | 3.3 mL | 696 μl |
| 1.5M Tris-HCl pH 8.8 | 2.5 mL | — |
| 0.5M Tris HCl pH 6.8 | — | 650 μl |
| 10% SDS | 100 μl | 100 μl |
| 10% APS | 50 μl | 50 μl |
| TEMED | 20 μl | 10 μl |
| Water (D) | 4.0 mL | 3.65 mL |

The Running Buffer is made by preparing 200 mL SDS Page Buffer (containing 288 g Glycine and 80 g Tris). 20 mL of SDS 10% buffer into 2000 mL of dH₂O will give the SDS buffer.

What is claimed is:

1. A dual qualitative and quantitative diagnostic immunoassay device for the detection of disease in a subject comprising:
(a) a dual quantitative and qualitative lateral flow immunoassay (LFIA) comprising:
(b) a central opening to allow contact of the LFIA with a biological sample;
(c) a first end of the LFIA comprises disease related biomarker detection following the principles of lateral flow immunoassay (LFIA) tests, wherein specific exosomal biomarkers for the disease under study are detected through the recognition of the antigens contained in the sample by a specific antibody stabilized in the strip; and
(d) a second end of the device comprises exosome identification and quantification based on a quantitative LFIA, wherein specific exosome biomarkers related to the disease under study are detected through the recognition of exosomal biomarkers contained in the biological sample by an antibody stabilized in the strip, wherein the visual read-out is accordingly adjusted so that the biomarker advancement distance is proportional to an amount of detected exosome from the biological sample.

2. The dual qualitative and quantitative diagnostic immunoassay of claim 1, wherein said exosomal biomarker comprises exosomal proteins, exosomal RNAs, or exosomal lncRNAs.

3. The dual qualitative and quantitative diagnostic immunoassay of claim 2, wherein said exosomal biomarker is an exosomal protein selected from the group consisting of CD9, CD81, CD63, CD147, CD235, CD41, COPB2 (coat complex subunit beta 2), PRKCB (protein kinase C beta), RHOC (ras homolog family member C), KRAS (KRAS proto-oncogene), CRP (C-reactive protein), CAPN2 (calpain 2), ECM1 (extracellular matrix protein 1), FGG (fibrinogen gamma chain), and MFAP4 (microfibril-associated glycoprotein 4 precursor).

4. The dual qualitative and quantitative diagnostic immunoassay of claim 2, wherein said exosomal biomarker is exosomal RNA selected from the group consisting of miR-15, miR-24-3p, hsa-miR-203-3p, hsa-miR-4482-3p, hsa-miR-44366b-3p, hsa-miR-190a-5p, miR-122-5p, SNORD33, AL732437.2, RNU2-29P, CDKN2B-AS1, and AL365184.1.

5. The dual qualitative and quantitative diagnostic immunoassay of claim 2, wherein said exosomal biomarker is exosomal lncRNA selected from the group consisting of LINC00657, LRRC75A-AS1, CRNDE, SNHG16, ZFAS1, and MALAT-1.

6. The dual qualitative and quantitative diagnostic immunoassay of claim 1, wherein said detecting comprises:
contacting said exosome with a labeled antibody that binds said exomsome; and
detecting the amount of labeled antibody associated with said exomsome immobilized on a substrate.

7. The dual qualitative and quantitative diagnostic immunoassay of claim 1, wherein said detecting comprises a competitive assay, said assay comprising the steps of:
(a) providing a first antibody that specifically binds said exosome;
(b) contacting said first antibody with a labeled exosome and said sample; and
(c) detecting the amount of labeled exosome that forms an exosome/antibody complex.

8. The dual qualitative and quantitative diagnostic immunoassay of claim 7, wherein said first antibody is immobilized on a solid substrate.

9. The dual qualitative and quantitative diagnostic immunoassay of claim 7, wherein said detecting comprises detecting the labeled exosome bound to said first antibody.

10. The dual qualitative and quantitative diagnostic immunoassay of claim 9, wherein said detecting comprises detecting the labeled exosome remaining unbound in solution.

11. The dual qualitative and quantitative diagnostic immunoassay of claim 1, wherein said detecting comprises Western Blotting utilizing an antibody that specifically binds to the exosome.

12. The dual qualitative and quantitative diagnostic immunoassay of claim 1, wherein the subject is a human.
